(19) United States
(12) Patent Application Publication
(10) Pub. No.: US 2025/0288218 A1
(43) Pub. Date: Sep. 18, 2025

(54) ELECTRONIC DEVICE AND METHOD FOR IDENTIFYING VASCULAR HEALTH CONDITION

(71) Applicant: Samsung Electronics Co., Ltd., Suwon-si (KR)

(72) Inventor: Hyunjun JUNG, Suwon-si (KR)

(21) Appl. No.: 19/226,560

(22) Filed: Jun. 3, 2025

Related U.S. Application Data
(63) Continuation of application No. PCT/KR2023/015775, filed on Oct. 12, 2023.

(30) Foreign Application Priority Data
Dec. 6, 2022 (KR) 10-2022-0169151
Jan. 18, 2023 (KR) 10-2023-0007630

(51) Int. Cl. A61B 5/026; A61B 5/024; A61B 5/0295

(52) U.S. Cl. CPC A61B 5/0261; A61B 5/02422; A61B 5/0295

(57) ABSTRACT

An electronic device may comprise at least one processor, a photoplethysmography (PPG) sensor. The at least one processor may be configured to cause the electronic device to: detect an external electronic device including a cuff; identify blood flow information via the PPG sensor; obtain compression time information of the cuff and relaxation time information of the cuff; obtain first perfusion index information based on the blood flow information and the compression time information; obtain second perfusion index information based on the blood flow information, the compression time information, and the relaxation time information; identify vascular health information based on a ratio of the first perfusion index information and the second perfusion index information; and display a notification to inform the user of a vascular health condition on the basis of the vascular health information.

### CROSS-REFERENCE TO RELATED APPLICATIONS

This application is a continuation International Application No. PCT/KR2023/015775 designating the United States, filed on Oct. 12, 2023, in the Korean Intellectual Property Receiving Office and claiming priority to Korean Patent application Nos. 10-2022-0169151, filed on Dec. 6, 2022, and 10-2023-0007630, filed on Jan. 18, 2023, in the Korean Intellectual Property Office, the disclosures of each of which are incorporated by reference herein in their entireties.

### BACKGROUND

### Field

The disclosure relates to an electronic device and a method for identifying a vascular health state.

### Description of Related Art

A service for identifying various health states through a wearable device is being provided. The service for identifying a health state may include a service that assists in health management by identifying a heart rate, blood pressure, and blood oxygen. The wearable device may provide a service for identifying information related to blood vessels of a user and identifying a health state based on the identified information.

The above-described information may be provided as related art for the purpose of

helping understanding of the present disclosure. No assertion or determination is made as to whether any of the above description may be applied as a prior art related to the present disclosure.

### SUMMARY

An electronic device according to example embodiments is provided. The electronic device may comprise: at least one processor, comprising processing circuitry, and a photoplethysmography (PPG) sensor. At least one processor, individually and/or collectively, may be configured to cause the electronic device to: detect an external electronic device comprising a cuff; identify blood flow rate information via the PPG sensor; obtain compression timing information of the cuff and relaxation timing information of the cuff; obtain first perfusion index information, based on the blood flow rate information and the compression timing information; obtain second perfusion index information, based on the blood flow rate information, the compression timing information, and the relaxation timing information; identify vascular health information based on a ratio of the second perfusion index information to the first perfusion index information; and display a notification indicating a vascular health state, based on the vascular health information.

A method performed by an electronic device according to example embodiments is provided. The method may comprise: detecting an external electronic device including a cuff; identifying blood flow rate information via a PPG sensor; obtaining compression timing information of the cuff and relaxation timing information of the cuff; obtaining first perfusion index information, based on the blood flow rate information and the compression timing information; obtaining second perfusion index information, based on the blood flow rate information, the compression timing information, and the relaxation timing information; identifying vascular health information based on a ratio of the second perfusion index information to the first perfusion index information; and displaying a notification indicating a vascular health state, based on the vascular health information.

A non-transitory computer-readable storage medium storing one or more programs according to example embodiments is provided. The one or more programs, when executed by at least one processor, comprising processing circuitry, individually and/or collectively, of an electronic device, may cause the electronic device to: detect an external electronic device including a cuff; identify blood flow rate information via the PPG sensor; obtain compression timing information of the cuff and relaxation timing information of the cuff; obtain first perfusion index information based on the blood flow rate information and the compression timing information; obtain second perfusion index information based on the blood flow rate information, the compression timing information, and the relaxation timing information; identify vascular health information based on a ratio of the second perfusion index information to the first perfusion index information; and display a notification indicating a vascular health state based on the vascular health information.

An electronic device according to example embodiments is provided. The electronic device may comprise: at least one processor, comprising processing circuitry, a photoplethysmography (PPG) sensor, and a cuff. The cuff may be configured to inflate by introduction of fluid. At least one processor, individually and/or collectively, may be configured to cause the electronic device to: identify blood flow rate information via the PPG sensor; obtain compression timing information of the cuff and relaxation timing information of the cuff; obtain first perfusion index information, based on the blood flow rate information and the compression timing information; obtain second perfusion index information, based on the blood flow rate information, the compression timing information, and the relaxation timing information; identify vascular health information based on a ratio of the second perfusion index information to the first perfusion index information; and display a notification indicating a vascular health state, based on the vascular health information.

A method performed by an electronic device according to example embodiments is provided. The method may comprise: identifying blood flow rate information via a photoplethysmography (PPG) sensor; obtaining compression timing information of a cuff and relaxation timing information of the cuff; obtaining first perfusion index information, based on the blood flow rate information and the compression timing information; obtaining second perfusion index information, based on the blood flow rate information, the compression timing information, and the relaxation timing information; identifying vascular health information based on a ratio of the second perfusion index information to the first perfusion index information; and displaying a notification indicating a vascular health state, based on the vascular health information.

A non-transitory computer-readable storage medium storing one or more programs according to example embodiments is provided. The one or more programs may include instructions that, when executed by at least one processor, comprising processing circuitry, individually and/or collectively, of an electronic device, cause the electronic device to: identify blood flow rate information via a photoplethysmography (PPG) sensor; obtain compression timing information of a cuff and relaxation timing information of the cuff; obtain first perfusion index information based on the blood flow rate information and the compression timing information; obtain second perfusion index information based on the blood flow rate information, the compression timing information, and the relaxation timing information; identify vascular health information based on a ratio of the second perfusion index information to the first perfusion index information; and display a notification indicating a vascular health state based on the vascular health information.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a block diagram illustrating an example electronic device in a network environment according to various embodiments;

FIGS. 2A and 2B are front and rear perspective views, respectively, of an example electronic device according to various embodiments;

FIG. 3 is an exploded perspective view of an example electronic device according to various embodiments;

FIG. 4 is a block diagram illustrating an example configuration of an electronic device according to various embodiments;

FIG. 5 is a diagram illustrating an example according to whether a cuff is included in an electronic device according to various embodiments;

FIG. 6 is a graph illustrating an example of a change in pressure and a change in blood flow rate information over time according to various embodiments;

FIG. 7 is a graph illustrating an example of a change in blood flow rate information and a change in perfusion index information over time according to various embodiments;

FIG. 8 is a diagram illustrating an example of a notification indicating a vascular health state according to various embodiments;

FIG. 9 is a flowchart illustrating an example operation of an electronic device to identify a vascular health state through a wearable device including a cuff according to various embodiments;

FIG. 10 is a flowchart illustrating an example operation of an electronic device to identify vascular health information according to various embodiments; and

FIG. 11 is a flowchart illustrating an example operation of an electronic device to identify a vascular health state based on a perfusion index according to various embodiments.

### DETAILED DESCRIPTION

Terms used in the present disclosure are used simply to describe various example embodiments, and are not intended to limit a range of the disclosure. A singular expression may include a plural expression unless the context clearly means otherwise. Terms used herein, including a technical or a scientific term, may have the same meaning as those generally understood by a person with ordinary skill in the art described in the present disclosure. Among the terms used in the present disclosure, terms defined in a general dictionary may be interpreted as identical or similar meaning to the contextual meaning of the relevant technology and are not interpreted as ideal or excessively formal meaning unless explicitly defined in the present disclosure. In some cases, even terms defined in the present disclosure may not be interpreted to exclude embodiments of the present disclosure.

In various embodiments of the present disclosure described below, a hardware approach will be described as an example. However, since the various embodiments of the present disclosure include technology that uses both hardware and software, the various embodiments of the present disclosure do not exclude a software-based approach.

A term referring to a cuff (e.g., a cuff, a fluid bag, and a tube), a term referring to a photoplethysmography (PPG) sensor (e.g., a PPG sensor and a blood flow measurement sensor), a term referring to a specified value (a reference value and a threshold value), and the like used in the following description are illustrated and described for convenience of explanation. Therefore, the present disclosure is not limited to terms to be described below, and another term having an equivalent technical meaning may be used. In addition, a term such as ‘ . . . unit,’ . . . device, ‘ . . . object’, and ‘ . . . structure’, and the like used below may refer, for example, to at least one shape structure or may refer, for example, to a unit processing a function.

In addition, in the present disclosure, the term ‘greater than’ or ‘less than’ may be used to determine whether a particular condition is satisfied or fulfilled, but this is only a description to express an example and does not exclude description of ‘greater than or equal to’ or ‘less than or equal to’. A condition described as ‘greater than or equal to’ may be replaced with ‘greater than’, a condition described as ‘less than or equal to’ may be replaced with ‘less than’, and a condition described as ‘greater than or equal to and less than’ may be replaced with ‘greater than and less than or equal to’. In addition, hereinafter, ‘A’ to ‘B’ refers to at least one of elements from A (including A) to B (including B).

Hereinafter, various example embodiments of the present disclosure are described with reference to the attached drawings. For convenience of description, components illustrated in the drawings may be exaggerated or reduced in size, and the present disclosure is not necessarily limited to what is illustrated.

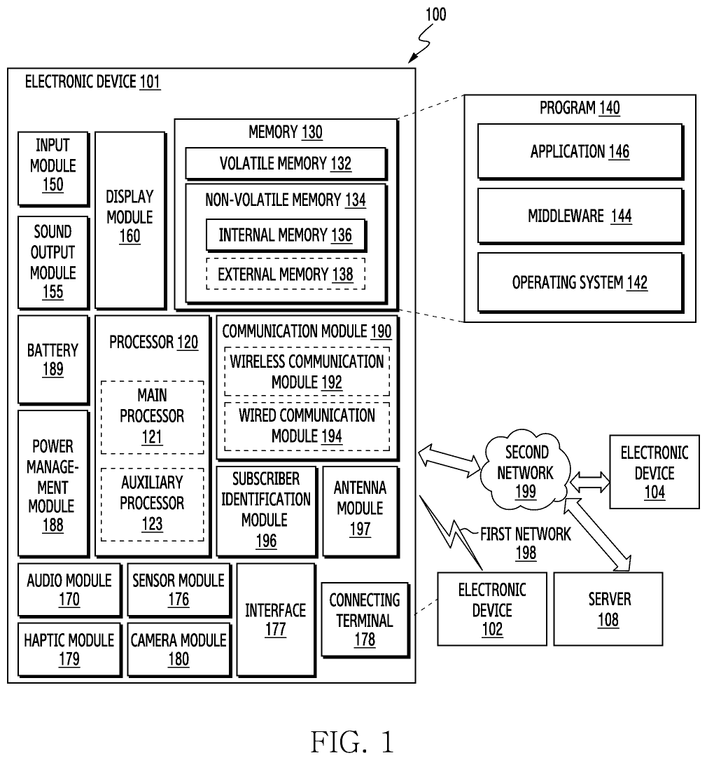

FIG. 1 is a block diagram illustrating an example electronic device 101 in a network environment 100 according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In various embodiments, at least one of the components (e.g., the connecting terminal 178) may be omitted from the electronic device 101, or one or more other components may be added in the electronic device 101. In various embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121. Thus, the processor 120 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, an HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mm Wave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating clement including a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In an embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

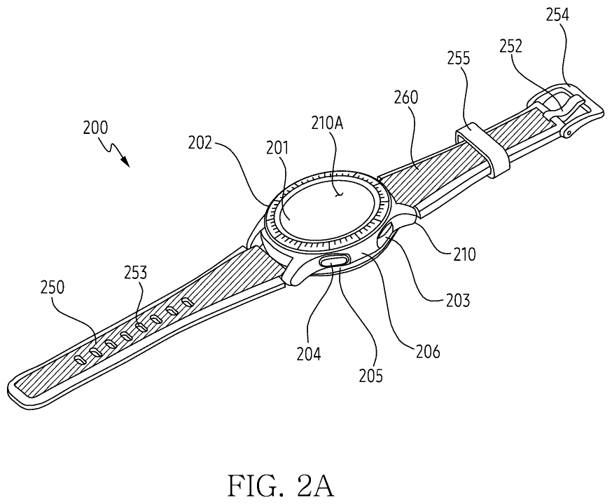

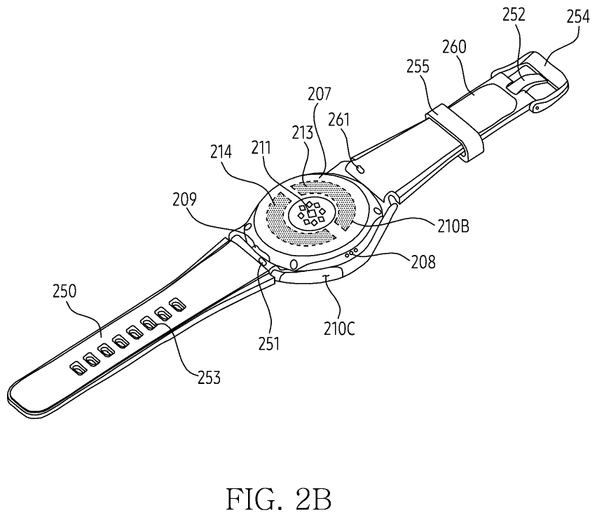

FIGS. 2A and 2B are front and rear perspective views, respectively, of an example electronic device according to various embodiments.

Referring to FIGS. 2A and 2B, an electronic device 200 (e.g., the electronic device 101 of FIG. 1) according to an embodiment may include a housing 210 including a first surface (or a front surface) 210A, a second surface (or a rear surface) 210B, and a lateral surface 210C surrounding a space between the first surface 210A and the second surface 210B, and fastening members 250 and 260 configured to connect to at least a portion of the housing 210 and detachably fasten the electronic device 200 to a part (e.g., a wrist or an ankle) of a user's body. In an embodiment (not illustrated), a housing may also refer to a structure forming a portion of the first surface 210A, the second surface 210B, and the lateral surface 210C of FIGS. 2A and 2B.

According to an embodiment, the first surface 210A may be formed by a front plate 201 (e.g., a glass plate or a polymer plate including various coating layers), in which at least a portion is substantially transparent. The second surface 210B may be formed by a rear plate 207 that is substantially opaque. For example, the rear plate 207 may be formed by coated or colored glass, ceramic, polymer, metal (e.g., aluminum, stainless steel (STS), or magnesium), or a combination of at least two of the materials. The lateral surface 210C may be coupled to the front plate 201 and the rear plate 207 and may be formed by a lateral bezel structure (or a “lateral member”) 206 including metal and/or polymer. In various embodiments, the rear plate 207 and the lateral bezel structure 206 may be formed integrally and may include the same material (e.g., a metal material such as aluminum). The fastening members 250 and 260 may be formed of various materials and shapes. An integral and a plurality of unit links may be formed to be movable with each other by a woven fabric, leather, rubber, urethane, metal, ceramic, or a combination of at least two of the materials.

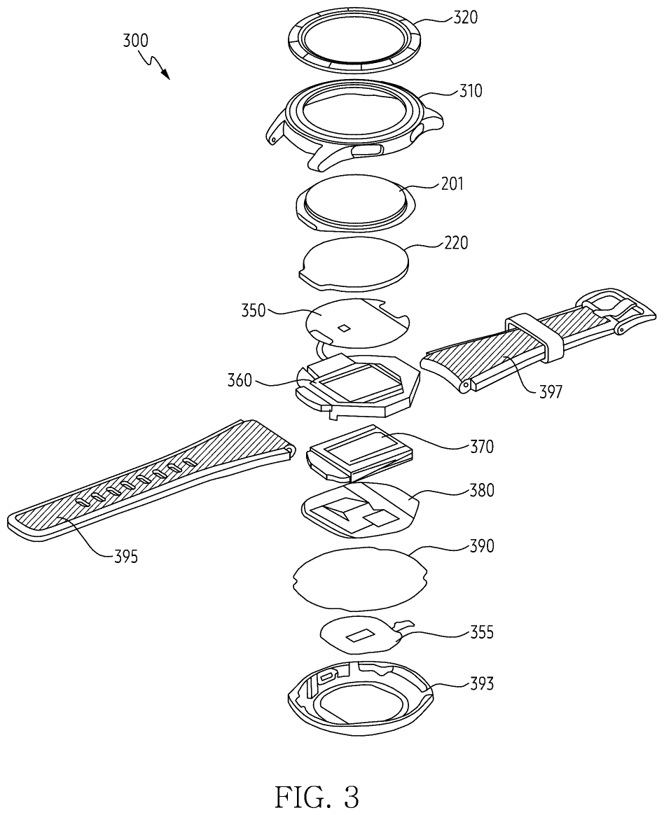

According to an embodiment, the electronic device 200 may include at least one of a display 220 (referencing FIG. 3), audio modules 205 and 208, a sensor module 211, key input devices 202, 203, and 204, and a connector hole 209. In various embodiments, the electronic device 200 may omit at least one of components (e.g., the key input devices 202, 203, and 204, the connector hole 209, or the sensor module 211) or may further include another component.

For example, the display 220 may be visible through a substantial portion of the front plate 201. A shape of the display 220 may correspond to a shape of the front plate 201 and may be of various shapes such as a circle, an oval, or a polygonal. The display 220 may be coupled to or disposed adjacent to touch detection circuitry, a pressure sensor capable of measuring an intensity (pressure) of a touch, and/or a fingerprint sensor.

The audio modules 205 and 208 may include a microphone hole 205 and a speaker hole 208. In the microphone hole 205, a microphone may be disposed inside to obtain an external sound, and in various embodiments, a plurality of microphones may be disposed to detect a direction of a sound. The speaker hole 208 may be used as an external speaker and a call receiver. In various embodiments, the speaker hole 208 and the microphone hole 205 may be implemented as one hole, or a speaker may be included without the speaker hole 208 (e.g., a piezoelectric speaker).

The sensor module 211 may generate an electrical signal or a data value corresponding to an internal operating state or an external environmental state of the electronic device 200. For example, the sensor module 211 may include a biometric sensor module 211 (e.g., a heart rate monitor (HRM) sensor) disposed on the second surface 210B of the housing 210. The electronic device 200 may further include at least one of a sensor module not illustrated, for example, a gesture sensor, a gyro sensor, a barometric sensor, a magnetic sensor, an acceleration sensor, a grip sensor, a color sensor, an infrared (IR) sensor, a biometric sensor, a temperature sensor, a humidity sensor, or an illuminance sensor.

The sensor module 211 may include electrode regions 213 and 214 forming a portion of a surface of the electronic device 200 and bio-signal detection circuitry (not illustrated) electrically connected to the electrode regions 213 and 214. For example, the electrode regions 213 and 214 may include a first electrode region 213 and a second electrode region 214 disposed on the second surface 210B of the housing 210. The sensor module 211 may be configured such that the electrode regions 213 and 214 obtain an electrical signal from the part of the user's body, and the bio-signal detection circuitry detects bio-information of the user based on the electrical signal.

The key input devices 202, 203, and 204 may include the wheel key 202 disposed on the first surface 210A of the housing 210 and rotatable in at least one direction, and/or the side key buttons 203 and 204 disposed on the lateral surface 210C of the housing 210. The wheel key may be a shape corresponding to the shape of the front plate 201. In an embodiment, the electronic device 200 may not include some or all of the key input devices 202, 203, and 204 mentioned above, and the key input devices 202, 203, and 204 that are not included may be implemented in another shape, such as a soft key, on the display 220. The connector hole 209 may include another connector hole (not illustrated) capable of accommodating a connector (e.g., a USB connector) for transmitting and receiving power and/or data with an external electronic device and capable of accommodating a connector for transmitting and receiving an audio signal with the external electronic device. For example, the electronic device 200 may further include a connector cover (not illustrated) that covers at least a portion of the connector hole 209 and blocks an inflow of an external foreign substance into the connector hole.

The fastening members 250 and 260 may be detachably fastened to at least a partial area of the housing 210 using locking members 251 and 261. The fastening members 250 and 260 may include one or more of a fixing member 252, a fixing member fastening hole 253, a band guide member 254, and a band fixing ring 255.

The fixing member 252 may be configured to fix the housing 210 and the fastening members 250 and 260 to the part (e.g., the wrist or the ankle) of the user's body. The fixing member fastening hole 253 may fix the housing 210 and the fastening members 250 and 260 to the part of the user's body by corresponding to the fixing member 252. The band guide member 254 may cause the fastening members 250 and 260 to be fastened in close contact with the part of the user's body, by being configured to limit a range of movement of the fixing member 252 when the fixing member 252 is fastened to the fixing member fastening hole 253. The band fixing ring 255 may limit a range of movement of the fastening members 250 and 260 in a state in which the fixing member 252 and the fixing member fastening hole 253 are fastened.

FIG. 3 is an exploded perspective view of an example electronic device according to various embodiments.

Referring to FIG. 3, an electronic device 300 (e.g., the electronic device 101 of FIG. 1 or the electronic device 200 of FIG. 2A to 2B) may include a lateral bezel structure 310, a wheel key 320 (e.g., the wheel key 202 of FIG. 2A), a front plate 201, a display 220, a first antenna 350, a second antenna 355, a support member 360 (e.g., a bracket), a battery 370, a printed circuit board 380, a sealing member 390, a rear plate 393 (e.g., the rear plate 207 of FIG. 2A), and fastening members 395 and 397 (e.g., the fastening members 250 and 260 of FIG. 2A). At least one of components of the electronic device 300 may be the same as or similar to at least one of the components of the electronic device 200 of FIG. 1 or FIGS. 2A to 2B, and an overlapping description will be omitted below. The support member 360 may be disposed inside the electronic device 300 to be connected to the lateral bezel structure 310 or may be formed integrally with the lateral bezel structure 310. For example, the support member 360 may be formed of a metal material and/or a non-metal material (e.g., polymer). In the support member 360, the display 220 may be coupled to a surface and the printed circuit board 380 may be coupled to another surface. A processor, memory, and/or an interface may be mounted on the printed circuit board 380. For example, the processor may include one or more of a central processing unit, a graphic processing unit (GPU), an application processor, a sensor processor, or a communication processor.

For example, the memory may include volatile memory or nonvolatile memory. For example, the interface may include a high definition multimedia interface (HDMI), a universal serial bus (USB) interface, an SD card interface, and/or an audio interface. For example, the interface may electrically or physically connect the electronic device 300 to an external electronic device, and may include a USB connector, an SD card/MMC connector, or an audio connector.

The battery 370 is a device for supplying power to the at least one component of the electronic device 300, and may include, for example, a non-rechargeable primary battery, a rechargeable secondary battery, or a fuel cell. For example, at least a portion of the battery 370 may be disposed on substantially the same plane as the printed circuit board 380. The battery 370 may be disposed integrally inside the electronic device 200 or may be disposed detachably from the electronic device 200.

The first antenna 350 may be disposed between the display 220 and the support member 360. For example, the first antenna 350 may include a near field communication (NFC) antenna, a wireless charging antenna, and/or a magnetic secure transmission (MST) antenna. For example, the first antenna 350 may perform short-range communication with an external device, or wirelessly transmit and receive power required for charging, and transmit a magnetic-based signal including a short-range communication signal or payment data. In an embodiment, an antenna structure may be formed by a portion of the lateral bezel structure 310 and/or the support member 360, or a combination thereof.

The second antenna 355 may be disposed between the printed circuit board 380 and the rear plate 393. For example, the second antenna 355 may include a near field communication (NFC) antenna, a wireless charging antenna, and/or a magnetic secure transmission (MST) antenna. For example, the second antenna 355 may perform short-range communication with the external device, or wirelessly transmit and receive the power required for charging, and transmit a magnetic-based signal including a short-range communication signal or payment data. In an embodiment, an antenna structure may be formed by a portion of the lateral bezel structure 310 and/or the rear plate 393, or a combination thereof.

The sealing member 390 may be positioned between the lateral bezel structure 310 and the rear plate 393. The sealing member 390 may be configured to block moisture and a foreign substance from inflowing into a space surrounded by the lateral bezel structure 310 and the rear plate 393 from the outside.

According to an embodiment, a wearable device (e.g., the electronic device 200 illustrated in FIGS. 2A and 2B) may be worn by a user and operated. The wearable device may provide a health service indicating a vascular health state by identifying vascular health information. The wearable device may be used to provide the health service indicating the vascular health state.

According to an embodiment, the wearable device may be used to identify the vascular health state according to the vascular health information and to guide early diagnosis and early treatment. For example, the wearable device may identify a degree of occurrence of reactive hyperemia and provide a guide for vascular health.

An operation of the wearable device according to the various example embodiments will be described in greater detail below. The electronic device described below may correspond to the electronic device 101 of FIG. 1 and/or the electronic device 200 of FIGS. 2A and 2B. The electronic device described below may be the wearable device. The wearable device may be various shapes that may be worn by the user, such as a smart watch, a smart band, and a smart ring.

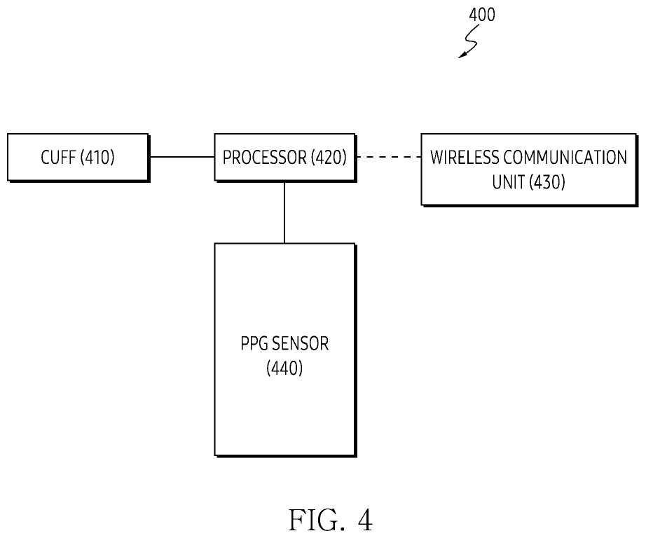

FIG. 4 is a block diagram illustrating an example configuration of an electronic device according to various embodiments.

Referring to FIG. 4, an electronic device 400 may correspond to the electronic device 101 of FIG. 1 and/or the electronic device 200 of FIGS. 2A and 2B. The electronic device 400 may include a cuff 410, a processor (e.g., including processing circuitry) 420 (e.g., the processor 120 of FIG. 1), a wireless communication unit (e.g., including wireless communication circuitry) 430 (e.g., the wireless communication module 192 of FIG. 1), and/or a photoplethysmography

(PPG) sensor 440 (e.g., the sensor module 176 of FIG. 1). According to an embodiment, the electronic device 400 may include at least one of the cuff 410, the processor 420, the wireless communication unit 430, and the PPG sensor 440. In other words, at least some of the cuff 410, the processor 420, the wireless communication unit 430, and the PPG sensor 440 may be omitted according to an embodiment.

According to an embodiment, the electronic device 400 may be a wearable device. The wearable device may be various shapes that may be worn by a user, such as a smart watch, a smart band, and a smart ring.

According to an embodiment, the cuff 410 may be included in the electronic device 400 or may be included in an external electronic device (e.g., a blood pressure monitor). When the cuff 410 is included in the external electronic device, the cuff 410 may not be included in the electronic device 400. The cuff 410 may include a device that inflates by introduction of fluid. The cuff 410 may apply pressure to an object by inflating by the introduced fluid. According to an embodiment, the object may be an arm on which the user wears the electronic device 400. A position of the cuff included in the external electronic device (e.g., the blood pressure monitor) may be closer to a heart than a position of the electronic device 400. A position of the cuff included in the electronic device 400 may be closer to the heart than a position of the PPG sensor 440. The cuff 410 may apply pressure to the arm on which the user wears the electronic device 400 to induce reactive hyperemia in blood vessels of the user. The reactive hyperemia may be a phenomenon in which blood flow significantly increases when the blood flow is resumed after blocking the blood flow over a short time period. The reactive hyperemia may be one of local regulatory functions of peripheral circulation. Blood vessels with reduced vascular elasticity due to a high degree of stiffness may have a low degree of a reactive hyperemia occurrence. Blood vessels with high vascular elasticity due to a low degree of stiffness may have a high degree of a reactive hyperemia occurrence. The electronic device may obtain vascular health information by identifying a degree of a reactive hyperemia occurrence. A vascular health state when a degree of vascular stiffness is high may be lower than a vascular health state when the vascular health information is low. The vascular health information may indicate a degree of vascular stiffness. Healthier blood vessels may have higher vascular elasticity. Blood vessels with higher vascular elasticity may be more capable of relaxing and contracting. Therefore, the blood vessels with high vascular elasticity may be better at circulating blood. The vascular elasticity may be reduced as cholesterol or blood clots are deposited on a wall of blood vessels. When a degree of vascular stiffness is low, risk of cardiovascular diseases such as arteriosclerosis, angina, and myocardial infarction may be high. In other words, the vascular health state when the degree of vascular stiffness is high may be lower than a vascular health state when the vascular health state information is low.

According to an embodiment, the processor 420 may include various processing circuitry and detect the external electronic device when the cuff 410 is included in the external electronic device. This is because when the cuff 410 is not included in the electronic device 400, vascular health information can be identified using the cuff 410 being included in the external electronic device. The processor 420 may receive information of the external electronic device for pairing. The processor 420 may transmit information of the electronic device to the external electronic device for the pairing. The processor 420 may display a notification to guide that the vascular health state may be checked based on detecting the external electronic device. The description of the processor 120 above may apply equally to the processor 420 here.

According to an embodiment, the processor 420 may display a notification for guiding a user wearing the electronic device 400 to wear the cuff included in the external electronic device on an arm on which the electronic device 400 is worn, based on detecting the external electronic device. For example, the electronic device 400 may display a notification such as ‘Please wear a blood pressure monitor on the arm wearing the smart watch’.

According to an embodiment, the processor 420 may identify blood flow rate information via the PPG sensor 440. The processor 420 may emit light to an object via the PPG sensor. The processor 420 may identify at least a portion of light reflected from the object. According to an embodiment, the object may be the arm on which the user wears the electronic device 400. The processor 420 may identify the blood flow rate information based on a ratio of an intensity of the emitted light to an intensity of the reflected light. The blood flow rate information may include the ratio of the intensity of the emitted light to the intensity of the reflected light. As the ratio increases, blood flow rate measured by the PPG sensor 440 may decrease. As the blood flow rate increases, the blood flow rate information may decrease. The blood flow rate information may correspond to an intensity of a PPG signal. According to an embodiment, the processor 420 may cause the user to maintain a measurement posture over a designated time period (e.g., approximately 10 seconds). The processor 420 may identify the blood flow rate information over the designated time period via the PPG sensor 440.

According to an embodiment, the processor 420 may display a notification to induce an operation of the cuff of the external electronic device. For example, the processor 420 may display a notification such as ‘Please operate the blood pressure monitor’.

According to an embodiment, the processor 420 may obtain compression timing information of the cuff and relaxation timing information of the cuff. For example, the processor 420 may receive a signal including the compression timing information and the relaxation timing information from the external electronic device. The relaxation timing may correspond to a timing at which a value corresponding to a maximum value of blood pressure measured by the external electronic device becomes equal to pressure applied by the cuff. For example, in order to identify the compression timing information and the relaxation timing information, the processor 420 may identify timing information of an interval at which a value of the blood flow rate information does not vibrate and increases. The processor 420 may identify start timing information of the increasing interval as the compression timing information. The processor 420 may identify end timing information of the increasing interval as the relaxation timing information.

In order to identify the increasing interval, the processor 420 may identify a range between a maximum value and a minimum value of the blood flow rate information during a threshold interval (e.g., an interval of 2 seconds from a target timing). In order to identify the increasing interval, the processor 420 may identify whether the blood flow rate exceeds the range between the maximum value and the minimum value. The processor 420 may identify an increase in the blood flow rate when the blood flow rate is identified to be greater than or equal to the maximum value. The processor 420 may identify a decrease in the blood flow rate when the blood flow rate is identified to be less than the minimum value.

The processor 420 may obtain first perfusion index information based on the blood flow rate information and the compression timing information. The first perfusion index information may be identified based on a value obtained by dividing an amplitude value of the blood flow rate information vibrating according to a pulse wave by a magnitude of the blood flow rate information before a compression timing. For example, the first perfusion index information may include the value obtained by dividing the amplitude value of the blood flow rate information vibrating according to the pulse wave by the magnitude of the blood flow rate information. The processor 420 may obtain second perfusion index information based on the blood flow rate information, the compression timing information, and the relaxation timing information. For example, the second perfusion index information may be identified based on a maximum value—over a certain time period after the relaxation timing—of a value obtained by dividing the amplitude of the blood flow rate information vibrating according to the pulse wave by the magnitude of the blood flow rate information and further dividing by the product of compression interval information.

According to an embodiment, the processor 420 may identify the vascular health information based on a ratio of the second perfusion index information to the first perfusion index information. For example, the vascular health information may include the ratio of the second perfusion index information to the first perfusion index information. According to an embodiment, the processor 420 may distinguish the vascular health state into three stages based on the vascular health information. For example, in a case that the ratio of the second perfusion index information to the first perfusion index information is less than a first threshold value, the processor 420 may identify vascular health information corresponding to a low degree of vascular health. The vascular health information may correspond to a degree of vascular stiffness. The vascular health information corresponding to the low degree of vascular health may indicate that the risk of cardiovascular diseases due to vascular stiffening, such as arteriosclerosis, angina, and myocardial infarction, is low. In a case that the ratio of the second perfusion index information to the first perfusion index information is greater than or equal to the first threshold value and less than a second threshold value, the processor 420 may identify vascular health information corresponding to a medium degree of vascular health. The vascular health information may correspond to a degree of vascular stiffness. The vascular health information corresponding to the medium degree of vascular health may indicate that the risk of cardiovascular diseases due to vascular stiffening, such as arteriosclerosis, angina, and myocardial infarction, is a medium degree.

In a case that the ratio of the second perfusion index information to the first perfusion index information is greater than or equal to the second threshold value, the processor 420 may identify vascular health information corresponding to a high degree of vascular health. The vascular health information may correspond to a degree of vascular stiffness. The vascular health information corresponding to the high degree of vascular health may indicate that the risk of cardiovascular diseases such as arteriosclerosis, angina, and myocardial infarction is high.

According to an embodiment, the processor 420 may distinguish the vascular health state into two stages based on the vascular health information. For example, in a case that the ratio of the second perfusion index information to the first perfusion index information is less than the first threshold value, the processor 420 may identify the vascular health information corresponding to the low degree of vascular health. In a case that the ratio of the second perfusion index information to the first perfusion index information is greater than or equal to the first threshold value, the processor 420 may identify the vascular health information corresponding to the high degree of vascular health.

According to an embodiment, the processor 420 may display a notification indicating the vascular health state based on the vascular health information. For example, based on the low degree of vascular health, a notification to guide that monitoring of the vascular health is necessary may be displayed. For another example, based on the high degree of vascular health, a notification to indicate a healthy state may be displayed.

According to an embodiment, the wireless communication unit 430 may include various wireless communication circuitry and detect the external electronic device including the cuff. Based on detecting the external electronic device, the wireless communication unit 430 may perform pairing between the electronic device 400 and the external electronic device. Through the pairing, the wireless communication unit 430 may obtain the compression timing information and the relaxation timing information from the external electronic device. Based on detecting the external electronic device, the wireless communication unit 430 may receive information of the external electronic device for the pairing. The wireless communication unit 430 may transmit information of the electronic device 400 to the external electronic device for the pairing. The information of the electronic device 400 and the information of the external electronic device may include information necessary for the electronic device 400 and the external electronic device to perform the pairing. For example, the information of the electronic device 400 may include a MAC address or an IP address. The information of the external electronic device may include a MAC address or an IP address. When direct pairing of the external electronic device (e.g., the blood pressure monitor) is impossible, a patch attached to the external electronic device may perform pairing with the electronic device 400. The electronic device 400, for which the pairing is completed, may transmit and receive data to the external electronic device. The processor 420 may store information on a communication method of the external electronic device according to a type of the external electronic device. For example, the processor 420 may register information on a frequency and a protocol used by the external electronic device as the information of the external electronic device.

According to an embodiment, the PPG sensor 440 may identify the blood flow rate information. The processor 420 may emit light to the object via the PPG sensor. The processor 420 may identify at least a portion of the light reflected from the object. According to an embodiment, the object may be the arm on which the user wears the electronic device 400. The processor 420 may identify the blood flow rate information based on the ratio between the intensity of the emitted light to the intensity of the reflected light. The blood flow rate information may include the ratio of the intensity of the emitted light to the intensity of the reflected light. As the ratio increases, the blood flow rate measured by the PPG sensor 440 may decrease. This is because, as the blood flow rate increases, an amount of light absorbed by the blood increases, and thus the intensity of the reflected light may decrease. As the blood flow rate increases, the blood flow rate information may decrease. The blood flow rate information may correspond to the intensity of the PPG signal.

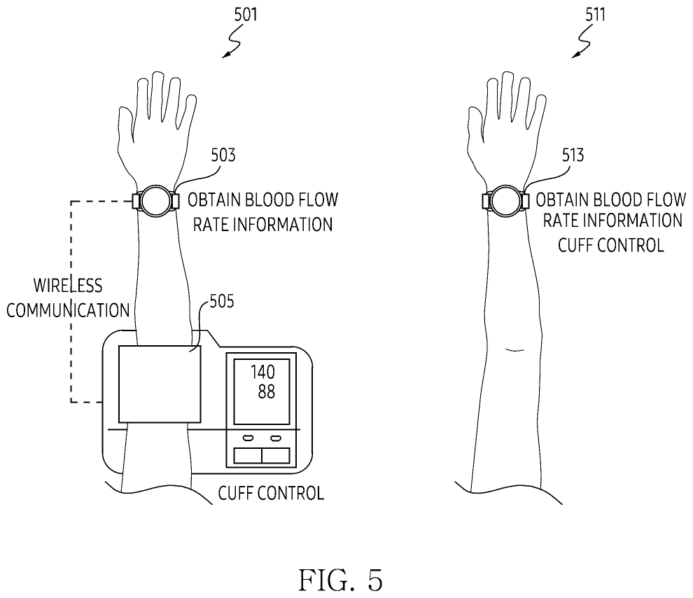

FIG. 5 is a diagram illustrating an example according to whether a cuff is included in an electronic device according to various embodiments.

Referring to FIG. 5, a use case 501 may be an embodiment in which a cuff is included in an external electronic device. A use case 511 may be an embodiment in which a cuff (e.g., the cuff 410 of FIG. 4) is included in the electronic device (e.g., the electronic device 200 of FIGS. 2A to 2B). In the use case 501, an electronic device 503 may check a vascular health state of a user by identifying blood flow rate information on the user. An external electronic device 505 may control the cuff included in the external electronic device 505. The electronic device 503 and the external electronic device 505 may transmit and/or receive data via wireless communication. In the use case 511, an electronic device 513 may control a cuff included in the electronic device 513 and check the vascular health state of the user by identifying the blood flow rate information on the user.

According to an embodiment, in the use case 501, the electronic device 503 may detect the external electronic device 505 (e.g., a blood pressure monitor). The electronic device 503 may display a notification to guide that the vascular health state may be checked based on detecting the external electronic device 505. For example, a notification such as ‘You may check the vascular health state using the blood pressure monitor’ may be displayed.

According to an embodiment the electronic device 503 may display a notification for guiding a user wearing the electronic device 503 to wear the cuff included in the external electronic device 505 on an arm of the user based on detecting the external electronic device 505. For example, the electronic device 503 may display a notification such as ‘Please wear the blood pressure monitor on the arm wearing the smart watch’. According to the notification, the user may insert the arm in the cuff included in the external electronic device 505.

According to an embodiment, the electronic device 503 may identify the blood flow rate information. While identifying the blood flow rate information, the electronic device 503 may display a notification to induce an operation of the cuff of the external electronic device 505. For example, the electronic device 503 may display a notification such as ‘Please operate the blood pressure monitor’. The user may operate the external electronic device 505 according to the notification. For example, the user may operate the blood pressure monitor according to the notification. While a cuff included in the blood pressure monitor is operated, the electronic device 503 may identify the blood flow rate information. The electronic device 503 may identify vascular dilation information by comparing blood flow rate information before and after the cuff is operated. When blood vessels are dilated to a threshold volume or more by a reactive hyperemia response after the cuff is operated, the electronic device 503 may identify vascular health information corresponding to a high degree of vascular health after the cuff is operated.

According to an embodiment, the electronic device 503 may obtain compression timing information of the cuff and relaxation timing information of the cuff from the external electronic device 505. The relaxation timing of the cuff may correspond to a timing at which a value corresponding to a maximum value of blood pressure measured by the external electronic device 505 becomes equal to pressure applied by the cuff. The electronic device 503 may transmit the compression timing information, which is a timing at which the operation of the cuff starts, to the electronic device 503. The external electronic device 505 may identify the timing at which the value corresponding to the maximum value of the blood pressure measured by the external electronic device 505 becomes equal to the pressure applied by the cuff as the relaxation timing information, and may transmit the relaxation timing information to the electronic device 503.

According to an embodiment, the electronic device 503 may identify the compression timing information of the cuff and the relaxation timing information of the cuff according to a value of the blood flow rate information. Even if the compression timing information and the relaxation timing information are not transmitted from the external electronic device 505, the electronic device 503 may identify the compression timing information and the relaxation timing information. For example, in order to identify the compression timing information and the relaxation timing information, the electronic device 503 may identify timing information of an interval at which the value of the blood flow rate information does not vibrate and increases. The electronic device 503 may identify start timing information of the increasing interval as the compression timing information. This is because when the cuff operates and blood flow is blocked for a short time period, the blood flow rate decreases and the value of the blood flow rate information (e.g., a PPG signal) increases. In addition, in the increasing interval, since the pressure applied by the cuff of the external electronic device 505 is greater than blood pressure of blood vessels in a systolic phase (e.g., systole, in other words, systolic blood pressure), a pulse wave generated by a heartbeat may not be formed. At least one processor (e.g., the processor 420 of FIG. 4) may identify end timing information of the increasing interval as the relaxation timing information. Since the pressure applied by the cuff of the external electronic device 505 is less than the blood pressure of the blood vessels in the systolic phase, the blood flow rate may increase and the value of the blood flow rate information may decrease. In addition, since the pressure applied by the cuff of the external electronic device 505 is less than the blood pressure of the blood vessels in the systolic phase, the blood flow rate information according to time may be identified in a form of the pulse wave.

The at least one processor 420 may obtain first perfusion index information based on the blood flow rate information and the compression timing information. The first perfusion index information may be identified based on a value obtained by dividing the amplitude value of the blood flow rate information vibrating according to the pulse wave by a magnitude of the blood flow rate information before the compression timing. For example, the first perfusion index information may include the value obtained by dividing the amplitude value of the blood flow rate information vibrating according to the pulse wave by the magnitude of the blood flow rate information before the compression timing. The at least one processor 420 may obtain second perfusion index information based on the blood flow rate information, the compression timing information, and the relaxation timing information. For example, the second perfusion index information may be identified based on a maximum value—over a certain time period after the relaxation timing—of a value obtained by dividing the amplitude of the blood flow rate information vibrating according to the pulse wave by the magnitude of the blood flow rate information and further dividing by the product of the compression interval information.

According to an embodiment, the at least one processor 420 may identify the vascular health information based on a ratio of the second perfusion index information to the first perfusion index information. For example, the vascular health information may include the ratio of the second perfusion index information to the first perfusion index information. According to an embodiment, the at least one processor 420 may distinguish the vascular health state into three stages based on the vascular health information. For example, in a case that the ratio of the second perfusion index information to the first perfusion index information is less than the first threshold value, the at least one processor 420 may identify vascular health information corresponding to a low degree of vascular health. In a case that the ratio of the second perfusion index information to the first perfusion index information is greater than or equal to the first threshold value and less than a second threshold value, the at least one processor 420 may identify vascular health information corresponding to a medium degree of vascular health. The vascular health information may include a degree of vascular stiffness. In a case that the ratio of the second perfusion index information to the first perfusion index information is greater than or equal to the second threshold value, the at least one processor 420 may identify vascular health information corresponding to a high degree of vascular health. According to an embodiment, the at least one processor 420 may distinguish the vascular health state into two stages based on the vascular health information. For example, in a case that the ratio of the second perfusion index information to the first perfusion index information is less than the first threshold value, the at least one processor 420 may identify the vascular health information corresponding to the low degree of vascular health. In a case that the ratio of the second perfusion index information to the first perfusion index information is greater than or equal to the first threshold value, the at least one processor 420 may indicate the high degree of vascular health.

According to an embodiment, in the use case 511, the electronic device 513 may display the notification to guide that the vascular health state may be checked. For example, a notification such as ‘You may check the vascular health state’ may be displayed. Since the cuff is included in the electronic device 513, the vascular health state may be identified by identifying the vascular health information without the external electronic device. Accordingly, the electronic device 513 may display a notification to guide that the vascular health state may be checked without the external electronic device.

According to an embodiment, the electronic device 513 may identify the blood flow rate information. While identifying the blood flow rate information, the electronic device 513 may operate the cuff. While the cuff is operated, the electronic device 513 may identify the blood flow rate information. The electronic device 513 may identify the vascular dilation information by comparing the blood flow rate information before and after the cuff is operated. When the blood vessels are dilated to a threshold volume or more by the reactive hyperemia response after the cuff is operated, the electronic device 513 may identify the vascular health information corresponding to the high degree of vascular health after the cuff operated.

According to an embodiment, the electronic device 513 may obtain the compression timing information of the cuff and the relaxation timing information of the cuff. The compression timing of the cuff may be a timing at which the cuff operates. The relaxation timing of the cuff may be a timing at which a value corresponding to a maximum value of blood pressure measured by the electronic device 513 becomes equal to the pressure applied by the cuff. The electronic device 513 may identify the timing at which the value corresponding to the maximum value of the blood pressure measured by the electronic device 513 becomes equal to the pressure applied by the cuff as the relaxation timing information.

According to an embodiment, the electronic device 513 may identify the compression timing information of the cuff and the relaxation timing information of the cuff according to the value of the blood flow rate information. The electronic device 513 may identify the compression timing information and the relaxation timing information without measuring blood pressure. For example, in order to identify the compression timing information and the relaxation timing information, the electronic device 513 may identify timing information of the interval at which the value of the blood flow rate information does not vibrate and increases. The electronic device 513 may identify the start timing information of the increasing interval as the compression timing information. This is because when the cuff operates and the blood flow is blocked for a short time period, the blood flow rate decreases and the value of the blood flow rate information (e.g., the PPG signal) increases. In addition, in the increasing interval, since the pressure applied by the cuff is greater than the blood pressure of the blood vessels in the systolic phase, the pulse wave generated by the heartbeat may not be formed. The electronic device 513 may identify the end timing information of the increasing interval as the relaxation timing information. Since the pressure applied by the cuff is less than the blood pressure of the blood vessels in the systolic phase, the blood flow rate may increase and the value of the blood flow rate information may decrease. In addition, since the pressure applied by the cuff is less than the blood pressure of the blood vessels in the systolic phase, the blood flow rate information according to time may be identified in the form of the pulse wave.

According to an embodiment, the electronic device 513 may obtain the first perfusion index information based on the blood flow rate information and the compression timing information. The electronic device 513 may obtain the second perfusion index information based on the blood flow rate information, the compression timing information, and the relaxation timing information. Since details regarding the first perfusion index and the second perfusion index are substantially the same as the use case 501, an overlapping description will be omitted below.

According to an embodiment, the electronic device 513 may identify the vascular health information based on the ratio of the second perfusion index information to the first perfusion index information. The second perfusion index information may be obtained. Since details regarding the vascular health information is substantially the same as the use case 501, an overlapping description will be omitted below.

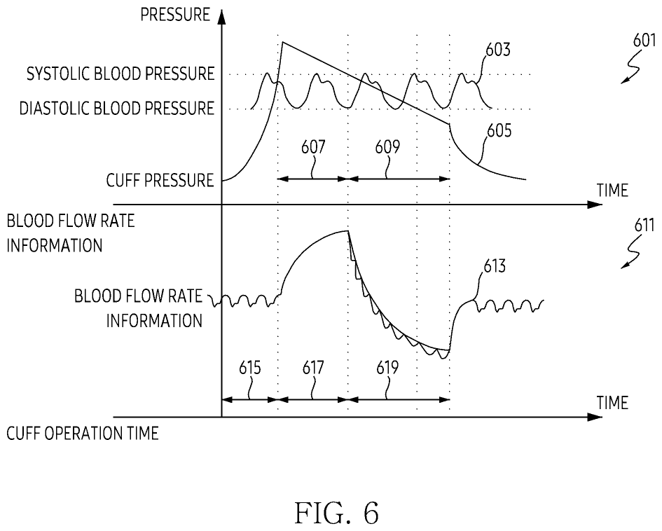

FIG. 6 is a graph illustrating an example of a change in pressure and a change in blood flow rate information over time according to various embodiments.

Referring to FIG. 6, a graph 601 may indicate a change in pressure that changes over time. An x-axis may be time. An y-axis may be pressure. A first line 603 may indicate blood pressure that changes periodically over time. A second line 605 may indicate pressure applied by a cuff that changes over time. In an interval 607, the pressure applied by the cuff may be greater than systolic blood pressure. In an interval 609, the pressure applied by the cuff may be less than the systolic blood pressure. A graph 611 may indicate a change in blood flow rate information over time. An x-axis may be time. An y-axis may be the blood flow rate information. A third line 613 may indicate a blood flow rate information value that changes over time. An interval 615 may be time before a compression timing. An interval 617 may be time after the compression timing and before a relaxation timing. An interval 619 may be time after the relaxation timing. The blood flow rate information value may be a ratio of an intensity of emitted light to an intensity of reflected light, measured by a PPG sensor.

According to an embodiment, in the first line 603, the blood pressure may vibrate in a form of a pulse wave by a heartbeat. In the second line 605, the pressure applied by the cuff may increase rapidly until the compression timing. The pressure applied by the cuff may gradually decrease linearly after the compression timing. A timing at which a value corresponding to a maximum value of identified blood pressure becomes equal to the pressure applied by the cuff may be identified as the relaxation timing. When the pressure applied by the cuff becomes less than a threshold value, the cuff may cease operation.

According to an embodiment, in the interval 615, blood flow rate information of the third line 613 may vibrate according to the pulse wave. This is because blood flow in blood vessels changes periodically by the heartbeat. The at least one processor (e.g., the processor 420 of FIG. 4) may identify first perfusion index information based on blood flow rate information before the compression timing. In the interval 617, the blood flow rate information of the third line 613 may increase without vibrating. The at least one processor 420 may identify start timing information of an interval at which a value of the blood flow rate information does not vibrate and increases as the compression timing information. This is because when the cuff operates and the blood flow is blocked for a short time period, the blood flow rate decreases and the value of blood flow rate information increases. In addition, in the interval 617, since the pressure applied by the cuff of the external electronic device (e.g., the external electronic device 505 of FIG. 5) is greater than blood pressure of the blood vessels in a systolic phase, the pulse wave generated by the heartbeat may not be formed. In the interval 619, the blood flow rate information of the third line 613 may vibrate and decrease. The at least one processor 420 may identify start timing information of the interval 619 as the relaxation timing information. The start timing information of the interval 619 may correspond to end timing information of the interval 617. Since the pressure applied by the cuff of the external electronic device 505 is less than the blood pressure of the blood vessels in the systolic phase, the blood flow rate may increase and the value of the blood flow rate information may decrease. In addition, since the pressure applied by the cuff of the external electronic device 505 is less than the blood pressure of the blood vessels in the systolic phase, the blood flow rate information according to time may be identified in the form of the pulse wave. The at least one processor 420 may identify second perfusion index information based on blood flow rate information over a certain time period after the relaxation timing.

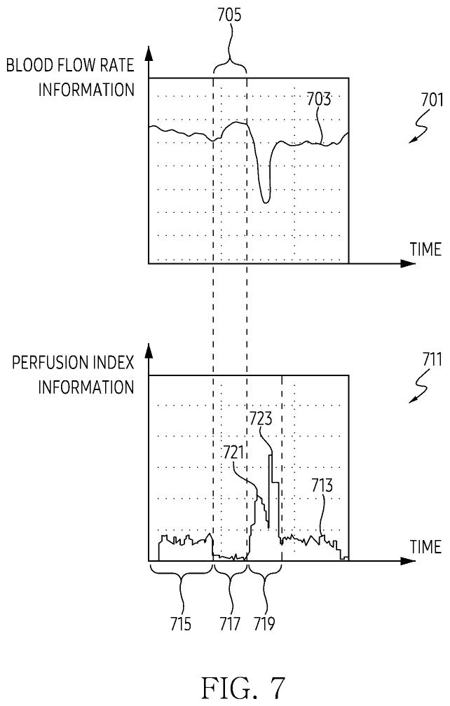

FIG. 7 is a graph illustrating an example of a change in blood flow rate information and a change in perfusion index information over time according to various embodiments.

Referring to FIG. 7, a graph 701 may indicate a change in blood flow rate information over time. An x-axis may be time. An y-axis may be blood flow rate information. A first line 703 may be blood flow rate information that changes according to an operation of a cuff over time. An interval 705 may be time at which a value of blood flow rate information does not vibrate and increases. The interval 705 may correspond to the interval 617 of FIG. 6. The interval 705 may be time between a compression timing of the cuff and relaxation timing of the cuff. A graph 711 may indicate a change in perfusion index information over time. An x-axis may be time. An y-axis may be perfusion index information. A second line 713 may be perfusion index information that changes according to the operation of the cuff over time. An interval 715 may be time before the compression timing. An interval 717 may be time after the compression timing and before the relaxation timing. An interval 719 may be time after the relaxation timing. The perfusion index information may indicate a magnitude of blood flow rate. For example, blood flow rate corresponding to large perfusion index information may be greater than blood flow rate corresponding to small perfusion index information.

In the interval 715, referring to the second line 713, perfusion index information before the compression timing may be first perfusion index information. The first perfusion index information may be identified based on a value obtained by dividing the amplitude value of the blood flow rate information vibrating according to the pulse wave by a magnitude of the blood flow rate information before the compression timing. For example, the first perfusion index information may include the value obtained by dividing the amplitude value of the blood flow rate information vibrating according to the pulse wave by the magnitude of the blood flow rate information.

In the interval 717, referring to the second line 713, perfusion index information at the compression timing may be lower than the perfusion index information in the interval 715. This is because pressure applied by the cuff is greater than systolic pressure of blood vessels, and blood flow through blood vessels in a portion where the cuff is worn is difficult.

In the interval 719, referring to the second line 713, at least one processor (e.g., the processor 420 of FIG. 4) may identify second perfusion index information based on blood flow rate information over a certain time period after the relaxation timing. The at least one processor 420 may obtain the second perfusion index information based on the blood flow rate information, the compression timing information, and the relaxation timing information. For example, the second perfusion index information may be identified based on a maximum value—over a certain time period after the relaxation timing—of a value obtained by dividing the amplitude of the blood flow rate information vibrating according to the pulse wave by the magnitude of the blood flow rate information and further dividing by the product of the compression interval information. For example, the second perfusion index information may be identified based on a maximum value—over a certain time period after the relaxation timing—of a value obtained by dividing the amplitude of the blood flow rate information vibrating according to the pulse wave by the magnitude of the blood flow rate information and normalizing it by the compression interval information. For example, the second perfusion index information may be identified based on an average value—over a certain time period after the relaxation timing—of a value obtained by dividing the amplitude of the blood flow rate information vibrating according to the pulse wave by the magnitude of the blood flow rate information and further dividing by the product of the compression interval information. For example, the second perfusion index may be a perfusion index information value of a first peak 721 in the interval 719. The compression interval information may include an interval of time after the compression timing and before the relaxation timing. For example, the compression interval information may be a time interval of the interval 717. This is because as a compression interval becomes longer, a degree of reactive hyperemia increases and thus a blood flow rate after the relaxation timing may increase. This is because as a length of the compression interval varies according to a type and a setting of a blood pressure monitor, which is an external electronic device, a value of the second perfusion index information may vary. Accordingly, the at least one processor 420 may normalize the second perfusion index information based on the compression interval information. A second peak 723 in the interval 719 may be an artifact.

According to an embodiment, the first perfusion index information in the interval 715 may be lower than the second perfusion index information in the interval 719. This is because blood flow is blocked for a short time period in the interval 717, and a reactive hyperemia phenomenon occurs in the interval 719. When the reactive hyperemia occurs, a blood flow rate may increase. Therefore, when the reactive hyperemia occurs in the interval 719, a perfusion index information value may also increase. The at least one processor 420 may identify vascular health information based on a ratio of the second perfusion index information to the first perfusion index information.

According to an embodiment, the vascular health information may include a vascular dilation index. The vascular dilation index may be the ratio of the second perfusion index information to the first perfusion index information. As the vascular dilation index increases, vascular elasticity of a user may increase. This is because the reactive hyperemia occurs more easily. The higher vascular elasticity may correspond to healthier blood vessels.

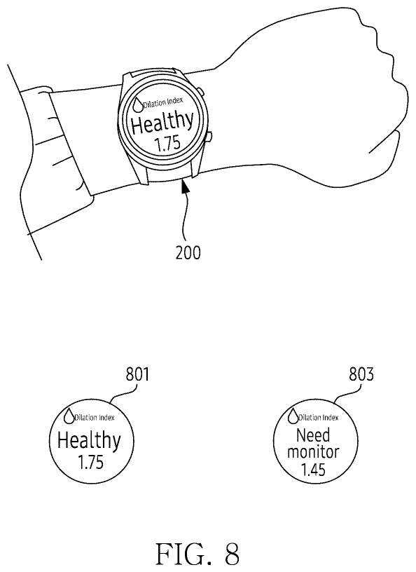

FIG. 8 is a diagram illustrating an example of a notification indicating a vascular health state according to various embodiments.

Referring to FIG. 8, an electronic device 200 may display a notification indicating a vascular health state based on vascular health information. The vascular health information may include the vascular dilation index of FIG. 7. A notification 801 may indicate a high degree of vascular health. A notification 803 may indicate a low degree of vascular health.

According to an embodiment, the notification 801 may display a ‘healthy’ indication to indicate the high degree of vascular health. The vascular dilation index may also be indicated. Since the vascular dilation index is described in FIG. 7, an overlapping description will be omitted below.

According to an embodiment, the notification 803 may display a ‘need monitor’ indication to indicate the low degree of vascular health. The vascular dilation index may also be indicated. The notification 803 may be an indication to recommend early diagnosis and management.

According to an embodiment, the electronic device 200 may display a notification indicating a measurement result for recent reference days (e.g., recent 3 days) in order to indicate the vascular health state. According to an embodiment, a notification indicating whether an outlier is identified by comparing the measurement result of the recent reference days may be displayed. According to an embodiment a notification may be displayed by dividing the vascular health state into three stages (e.g., very good, continuous observation, and attention required). The electronic device 200 may visualize the notification by changing colors according to the three stages. According to an embodiment, the electronic device 200 may detect an outlier when an identification value of the vascular health information is outside a confidence interval compared to an identification value of the vascular health information for the recent reference days. According to an embodiment, the electronic device 200 may divide the vascular health state according to a interval based on a clinically obtained data interval. According to an embodiment the electronic device 200 may display a notification to guide remeasurement when an abnormal measurement is detected. The electronic device 200 may delete the abnormal measurement when the vascular health information at time of the remeasurement is in a normal measurement range. According to an embodiment, if the vascular health has not been identified over the recent reference days (e.g., recent 5 days) when a blood pressure monitor in the vicinity is detected, the electronic device 200 may display a notification to recommend vascular health identification. According to an embodiment, when the blood pressure monitor is detected, the electronic device 200 may identify the vascular health information without indicating the notification recommending the identification of the vascular health information. For example, when a user uses the blood pressure monitor, the vascular health information may be identified using an operation of the blood pressure monitor without indicating the notification related to the vascular health information. The electronic device 200 may indicate a notification indicating current vascular health information to the user when a frequency of the abnormal measurement over the recent reference days is greater than or equal to a threshold frequency and dispersion of the abnormal measurements is less than a threshold dispersion.

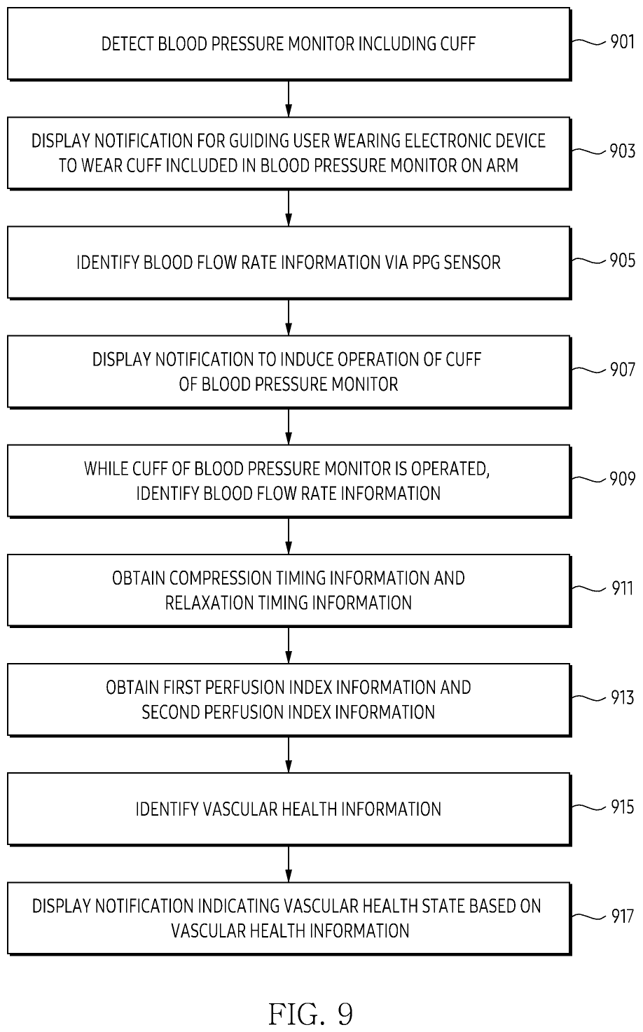

FIG. 9 is a flowchart illustrating an example operation of an electronic device to identify a vascular health state through a wearable device including a cuff according to various embodiments.

In the following example, each of operations may be performed sequentially, but is not necessarily performed sequentially. For example, an order of each of the operations may be changed, and at least two operations may be performed in parallel.

According to an embodiment, operations 901 to 917 may be understood to be performed in a processor (e.g., the processor 420 of FIG. 4) of the electronic device (e.g., the electronic device 400 of FIG. 4).

Referring to FIG. 9, in operation 901, according to an embodiment, at least one processor (e.g., the processor 420 of FIG. 4) may detect a blood pressure monitor (e.g., the external electronic device 505 of FIG. 5) including a cuff (e.g., the cuff 410 of FIG. 4). This is because when the cuff 410 is not included in the electronic device (e.g., the electronic device 200 of FIGS. 2A to 2B), vascular health information may be identified, only when the cuff 410 included in the blood pressure monitor is used. The at least one processor 420 may receive information of the blood pressure monitor for pairing. The at least one processor 420 may transmit information of the electronic device 200 to the blood pressure monitor for the pairing. The information of the electronic device 200 and the information of the blood pressure monitor may include information necessary for the electronic device 200 and the blood pressure monitor to perform the pairing. For example, the information of the electronic device 200 may include a MAC address or an IP address. The information of the blood pressure monitor may include a MAC address or an IP address. When direct pairing of the blood pressure monitor is impossible, a patch attached to the blood pressure monitor may perform pairing with the electronic device 200. The electronic device 200, for which the pairing is completed, may transmit and receive data to the blood pressure monitor. The at least one processor 420 may store information on a communication method of the blood pressure monitor according to a type of the blood pressure monitor. For example, the at least one processor 420 may register information on a frequency and a protocol used by the blood pressure monitor as the information of the blood pressure monitor.

According to an embodiment, the at least one processor 420 may display a notification to guide that the vascular health state may be checked based on detecting the blood pressure monitor. For example, a notification such as ‘You may check the vascular health state using the blood pressure monitor’ may be displayed.

In operation 903, according to an embodiment, the at least one processor 420 may display a notification for guiding a user wearing the electronic device 200 to wear the cuff included in the blood pressure monitor on an arm of the user. For example, the electronic device 200 may display a notification such as ‘Please wear the blood pressure monitor on the arm wearing the smart watch’. According to an embodiment, the user may insert the arm in the cuff included in the blood pressure monitor. A position of the cuff may be closer to a heart than a position of the electronic device 200.

In operation 905, according to an embodiment, the at least one processor 420 may identify blood flow rate information via a PPG sensor (e.g., the PPG sensor 440 of FIG. 4). The PPG sensor 440 may identify the blood flow rate information. The at least one processor 420 may emit light to an object via the PPG sensor 440. The at least one processor 420 may identify light reflected from the object. According to an embodiment, the object may be the arm on which the electronic device 400 is worn by the user. The at least one processor 420 may identify the blood flow rate information based on a ratio between an intensity of the emitted light to an intensity of the reflected light. The blood flow rate information may include the ratio of the intensity of the emitted light to the intensity of the reflected light. As the ratio increases, blood flow rate measured by the PPG sensor 440 may decrease. This is because, as the blood flow rate increases, an amount of light absorbed by the blood increases, and thus the intensity of the reflected light may decrease. As the blood flow rate increases, the blood flow rate information may decrease. The blood flow rate information may correspond to an intensity of a PPG signal.

In operation 907, according to an embodiment, the at least one processor 420 may display a notification to induce an operation of the cuff of the blood pressure monitor. The at least one processor 420 may display a notification such as ‘Please operate the blood pressure monitor’.

In operation 909, the at least one processor 420 may identify the blood flow rate information while the cuff of the blood pressure monitor is operated. This is because the electronic device 200 may identify vascular dilation information by comparing the blood flow rate information before and after the cuff is operated.

In operation 911, the at least one processor 420 may obtain compression timing information and relaxation timing information. According to an embodiment, the at least one processor 420 may receive a signal including the compression timing information and the relaxation timing information from the blood pressure monitor. The relaxation timing may be a timing at which a value corresponding to a maximum value of blood pressure measured by the blood pressure monitor becomes equal to pressure applied by the cuff 410. According to an embodiment, in order to identify the compression timing information and the relaxation timing information, the at least one processor 420 may identify timing information of an interval at which a value of the blood flow rate information does not vibrate and increases. The at least one processor 420 may identify start timing information of the increasing interval as the compression timing information. The at least one processor 420 may identify end timing information of the increasing interval as the relaxation timing information.

In operation 913, the at least one processor 420 may obtain first perfusion index information and second perfusion index information. The at least one processor 420 may obtain the first perfusion index information based on the blood flow rate information and the compression timing information. The first perfusion index information may be identified based on a value obtained by dividing the amplitude value of the blood flow rate information vibrating according to the pulse wave by a magnitude of the blood flow rate information before a compression timing. For example, the first perfusion index information may include the value obtained by dividing the amplitude value of the blood flow rate information vibrating according to the pulse wave by the magnitude of the blood flow rate information. The at least one processor 420 may obtain the second perfusion index information based on the blood flow rate information, the compression timing information, and the relaxation timing information. For example, the second perfusion index information may be identified based on a maximum value—over a certain time period after the relaxation timing—of a value obtained by dividing the amplitude of the blood flow rate information vibrating according to the pulse wave by the magnitude of the blood flow rate information and normalizing it by the compression interval information. For example, the second perfusion index information may be identified based on an average value—over a certain time period after the relaxation timing—of a value obtained by dividing the amplitude of the blood flow rate information vibrating according to the pulse wave by the magnitude of the blood flow rate information and further dividing by the product of the compression interval information. The compression interval information may include an interval of time after the compression timing and before the relaxation timing. This is because as a compression interval becomes longer, a degree of reactive hyperemia increases and blood flow rate after the relaxation timing may increase. This is because as a length of the compression interval varies according to a type and/or a setting of the blood pressure monitor which is an external electronic device, a value of the second perfusion index information may vary. Accordingly, the at least one processor 420 may normalize the second perfusion index information based on the compression interval information.

In operation 915, the at least one processor 420 may identify vascular health information. The vascular health information may include a vascular dilation index. According to an embodiment, the at least one processor 420 may identify the vascular health information based on a ratio of the second perfusion index information to the first perfusion index information. For example, the vascular health information may include the ratio of the second perfusion index information to the first perfusion index information. According to an embodiment, the at least one processor 420 may distinguish the vascular health state into three stages based on the vascular health information. For example, in a case that the ratio of the second perfusion index information to the first perfusion index information is less than the first threshold value, the at least one processor 420 may identify vascular health information corresponding to a low degree of vascular health. In a case that the ratio of the second perfusion index information to the first perfusion index information is greater than or equal to the first threshold value and less than a second threshold value, the at least one processor 420 may identify vascular health information corresponding to a medium degree of vascular health. In a case that the ratio of the second perfusion index information to the first perfusion index information is greater than or equal to the second threshold value, the at least one processor 420 may identify vascular health information corresponding to a high degree of vascular health. According to an embodiment, the at least one processor 420 may distinguish the vascular health state into two stages based on the vascular health information. For example, in a case that the ratio of the second perfusion index information to the first perfusion index information is less than the first threshold value, the at least one processor 420 may identify the vascular health information corresponding to the low degree of vascular health. In a case that the ratio of the second perfusion index information to the first perfusion index information is greater than or equal to the first threshold value, the at least one processor 420 may identify the vascular health information corresponding to the high degree of vascular health.

In operation 917, the at least one processor 420 may display a notification indicating the vascular health state based on the vascular health information. For example, based on the low degree of vascular health, a notification to guide that monitoring of the vascular health is necessary may be displayed. For another example, based on the high degree of vascular health, a notification to indicate a healthy state may be displayed. A description of the notification is described in greater detail above with reference to FIG. 8.

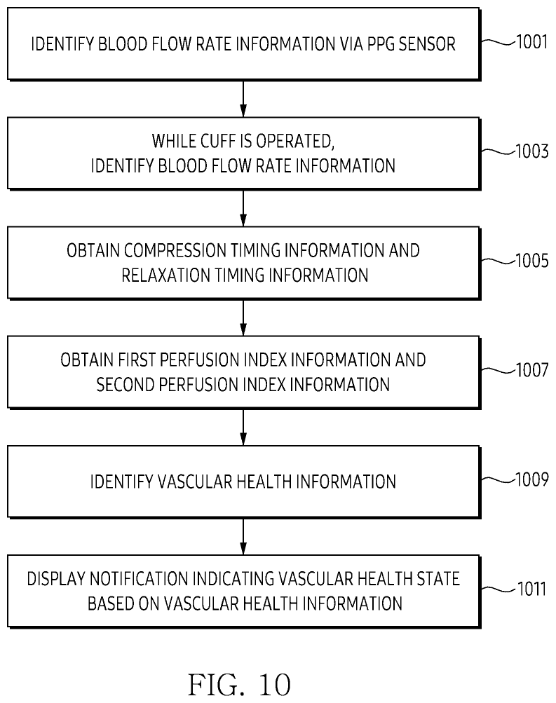

FIG. 10 is a flowchart illustrating an example operation of an electronic device to identify vascular health information according to various embodiments.

In the following example, each of operations may be performed sequentially, but is not necessarily performed sequentially. For example, an order of each of the operations may be changed, and at least two operations may be performed in parallel.

According to an embodiment, operations 1001 to 1011 may be understood to be performed in a processor (e.g., the processor 420 of FIG. 4) of the electronic device (e.g., the electronic device 400 of FIG. 4).

Referring to FIG. 10, in operation 1001, according to an embodiment, at least one processor (e.g., the processor 420 of FIG. 4) may identify blood flow rate information via a PPG sensor (e.g., the PPG sensor 440 of FIG. 4). Since the electronic device (e.g., the electronic device 200 of FIGS. 2A to 2B) includes a cuff (e.g., the cuff 410 of FIG. 4), the electronic device 200 may identify a vascular health state without detecting whether a blood pressure monitor (e.g., the external electronic device 505 of FIG. 5) is close. The electronic device 200 may display a notification to guide that the vascular health state may be checked. For example, a notification such as ‘You may check the vascular health state’ may be displayed. A position of the cuff may be closer to a heart than a position of the PPG sensor 440.

In operation 1003, the at least one processor 420 may identify the blood flow rate information while the cuff 410 is operated. This is because the electronic device 200 may identify vascular dilation information by comparing the blood flow rate information before and after the cuff 410 is operated. When the blood vessels are dilated to a threshold volume or more by the reactive hyperemia response after the cuff 410 is operated, the electronic device 200 may identify the vascular health information corresponding to the high degree of vascular health after the cuff 410 is operated.

In operation 1005, the at least one processor 420 may obtain compression timing information and relaxation timing information.

According to an embodiment, the electronic device 200 may obtain the compression timing information of the cuff 410 and the relaxation timing information of the cuff 410. The compression timing of the cuff 410 may be a timing at which the cuff 410 operates. The relaxation timing of the cuff 410 may be a timing at which a value corresponding to a maximum value of blood pressure measured by the electronic device 200 becomes equal to pressure applied by the cuff 410. The electronic device 200 may identify the timing at which the value corresponding to the maximum value of the blood pressure measured by the electronic device 200 becomes equal to the pressure applied by the cuff 410 as the relaxation timing information.

According to an embodiment, the electronic device 200 may identify the compression timing information of the cuff 410 and the relaxation timing information of the cuff 410 according to a value of the blood flow rate information. The electronic device 200 may identify the compression timing information and the relaxation timing information without measuring blood pressure. For example, in order to identify the compression timing information and the relaxation timing information, the electronic device 200 may identify timing information of an interval at which the value of the blood flow rate information does not vibrate and increases. The electronic device 200 may identify start timing information of the increasing interval as the compression timing information. This is because when the cuff 410 operates and blood flow is blocked for a short time period, blood flow rate decreases and the value of the blood flow rate information (e.g., a PPG signal) increases. In addition, in the increasing interval, since the pressure applied by the cuff 410 is greater than blood pressure of the blood vessels in a systolic phase, a pulse wave generated by a heartbeat may not be formed. The electronic device 200 may identify end timing information of the increasing interval as the relaxation timing information. Since the pressure applied by the cuff 410 is less than the blood pressure of the blood vessels in the systolic phase, the blood flow rate may increase and the value of the blood flow rate information may decrease.

In addition, since the pressure applied by the cuff 410 is less than the blood pressure of the blood vessels in a systolic phase, the blood flow rate information according to time may be identified in a form of the pulse wave.

In operation 1007, the at least one processor 420 may obtain first perfusion index information and second perfusion index information. The at least one processor 420 may obtain the first perfusion index information based on the blood flow rate information and the compression timing information. The first perfusion index information may be identified based on a value obtained by dividing the amplitude value of the blood flow rate information vibrating according to the pulse wave by a magnitude of the blood flow rate information before the compression timing. For example, the first perfusion index information may include the value obtained by dividing the amplitude value of the blood flow rate information vibrating according to the pulse wave by the magnitude of the blood flow rate information. The at least one processor 420 may obtain second perfusion index information based on the blood flow rate information, the compression timing information, and the relaxation timing information. For example, the second perfusion index information may be identified based on a maximum value—over a certain time period after the relaxation timing—of a value obtained by dividing the amplitude of the blood flow rate information vibrating according to the pulse wave by the magnitude of the blood flow rate information and further dividing by the product of the compression interval information.

In operation 1009, the at least one processor 420 may identify the vascular health information. The vascular health information may include a vascular dilation index. The vascular health information may indicate a degree of vascular stiffness. According to an embodiment, the at least one processor 420 may identify the vascular health information based on a ratio of the second perfusion index information to the first perfusion index information. For example, the vascular health information may include the ratio of the second perfusion index information and the first perfusion index information. According to an embodiment, the at least one processor 420 may distinguish the vascular health state into three stages based on the vascular health information. According to an embodiment, the at least one processor 420 may distinguish the vascular health state into two stages based on the vascular health information. Since details regarding the vascular health information are described in FIG. 9, an overlapping description will be omitted below.

In operation 1011, the at least one processor 420 may display a notification indicating the vascular health state based on the vascular health information. For example, based on a low degree of vascular health, a notification to guide that monitoring of vascular health is necessary may be displayed. For another example, based on a high degree of vascular health, a notification to indicate a healthy state may be displayed. A description of the notification is described in greater detail above with reference to FIG. 8.

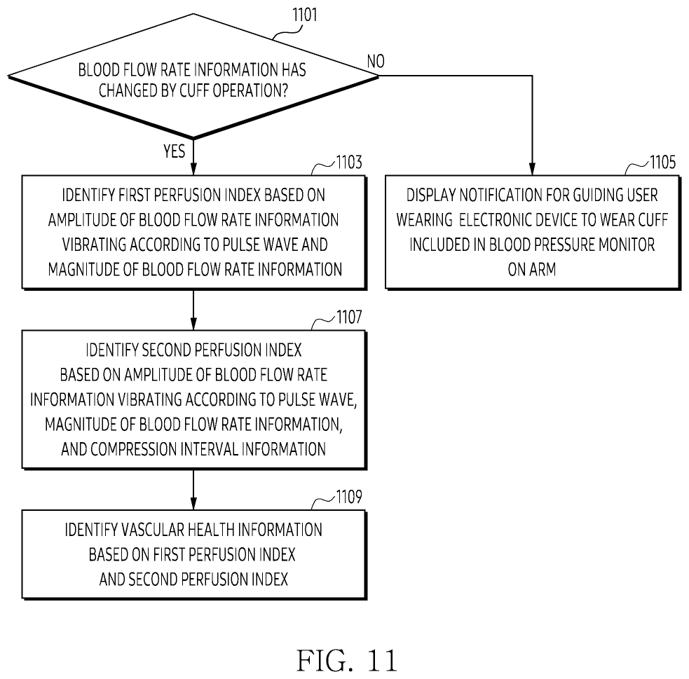

FIG. 11 is a flowchart illustrating an example operation of an electronic device to identify a vascular health state based on a perfusion index according to various embodiments.

In the following example, each of operations may be performed sequentially, but is not necessarily performed sequentially. For example, an order of each of the operations may be changed, and at least two operations may be performed in parallel.

According to an embodiment, operations 1101 to 1109 may be understood to be performed in a processor (e.g., the processor 420 of FIG. 4) of the electronic device (e.g., the electronic device 400 of FIG. 4).

Referring to FIG. 11, in operation 1101, at least one processor (e.g., the processor 420 of FIG. 4) may identify whether blood flow rate information has been changed by a cuff operation.

The at least one processor 420 may start measuring the blood flow rate information by an input of a user. The at least one processor 420 may display a notification to induce an operation of a cuff of a blood pressure monitor. When the blood flow rate information has changed by the cuff operation, the at least one processor 420 may perform the operation 1103. When the blood flow rate information has not changed by the cuff operation, the at least one processor 420 may perform the operation 1105. This is because when the blood flow rate information does not change even if the cuff is in operation, it may be a situation in which the blood pressure monitor is not worn on an arm of a user wearing the electronic device (e.g., the electronic device 200 of FIGS. 2A to 2B).

In operation 1103, the at least one processor 420 may identify a first perfusion index based on an amplitude of the blood flow rate information vibrating according to a pulse wave and a magnitude of the blood flow rate information. For example, the first perfusion index information may include a value obtained by dividing the amplitude value of the blood flow rate information vibrating according to the pulse wave by the magnitude of the blood flow rate information.

In operation 1105, the at least one processor 420 may display a notification for guiding a user wearing the electronic device to wear a cuff included in the blood pressure monitor on an arm of the user. This is because, when the blood flow rate information does not change even if the cuff is in operation, it may be a situation in which the blood pressure monitor is not worn on the arm of the user wearing the electronic device 200. When the blood pressure monitor is not worn on the arm of the user wearing the electronic device 200, the electronic device 200 may not be able to identify vascular health information as reactive hyperemia is difficult to occur by the operation of the cuff. Accordingly, the electronic device 200 may display a notification guiding the user wearing the electronic device 200 to wear the blood pressure monitor on the arm.

In operation 1107, the at least one processor 420 may identify a second perfusion index based on the amplitude of the blood flow rate information vibrating according to the pulse wave, the magnitude of the blood flow rate information, and compression interval information. For example, the second perfusion index information may be identified based on a maximum value—over a certain time period after a relaxation timing—of a value obtained by dividing the amplitude of the blood flow rate information vibrating according to the pulse wave by the magnitude of the blood flow rate information and normalizing it by the compression interval information. For example, the second perfusion index information may be identified based on an average value—over a certain time period after the relaxation timing—of a value obtained by dividing the amplitude of the blood flow rate information vibrating according to the pulse wave by the magnitude of the blood flow rate information and further dividing by the product of the compression interval information. The compression interval information may include an interval of time after a compression timing and before the relaxation timing. This is because as a compression interval becomes longer, a degree of reactive hyperemia increases and blood flow rate after the relaxation timing may increase. This is because as a length of the compression interval varies according to a type and/or a setting of the blood pressure monitor, which is an external electronic device, and a value of the second perfusion index information may vary. Accordingly, the at least one processor 420 may normalize the second perfusion index information based on the compression interval information.

In operation 1109, the at least one processor 420 may identify the vascular health information based on the first perfusion index and the second perfusion index. The vascular health information may include a vascular dilation index. According to an embodiment, the at least one processor 420 may identify the vascular health information based on a ratio of the second perfusion index information to the first perfusion index information. For example, the vascular health information may include the ratio of the second perfusion index information to the first perfusion index information. According to an embodiment, the at least one processor 420 may distinguish the vascular health state into three stages based on the vascular health information. According to an embodiment, the at least one processor 420 may distinguish the vascular health state into two stages based on the vascular health information. Since details regarding the vascular health information are described in FIG. 9, an overlapping description will be omitted below.

According to embodiments of the present disclosure, the electronic device (e.g., the electronic device 200 of FIGS. 2A to 2B) may identify the vascular health information by inducing the reactive hyperemia without performing prolonged occlusion for approximately 2 minutes to approximately 5 minutes.

As described above, an electronic device 101, 200, or 503 according to an example embodiment may comprise at least one processor 120 or 420 and a photoplethysmography (PPG) sensor 440. The at least one processor 120 or 420 may detect an external electronic device 505 comprising a cuff. The at least one processor 120 or 420 may identify blood flow rate information via the PPG sensor 440. The at least one processor 120 or 420 may obtain compression timing information of the cuff and relaxation timing information of the cuff. The at least one processor 120 or 420 may obtain first perfusion index information, based on the blood flow rate information and the compression timing information. The at least one processor 120 or 420 may obtain second perfusion index information, based on the blood flow rate information, the compression timing information, and the relaxation timing information. The at least one processor 120 or 420 may identify vascular health information based on a ratio of the second perfusion index information to the first perfusion index information. The at least one processor 120 or 420 may display a notification indicating a vascular health state, based on the vascular health information.

According to an example embodiment, in order to identify the vascular health information, the at least one processor 120 or 420 may identify vascular health information corresponding to a low degree of vascular health, in a case that the ratio of the second perfusion index information to the first perfusion index information is less than a first threshold value. In order to identify the vascular health information, the at least one processor 120 or 420 may identify vascular health information corresponding to a medium degree of vascular health, in a case that the ratio of the second perfusion index information to the first perfusion index information is greater than or equal to the first threshold value and less than a second threshold value. In order to identify the vascular health information, the at least one processor 120 or 420 may identify vascular health information corresponding to a high degree of vascular health, in a case that the ratio of the second perfusion index information to the first perfusion index information is greater than or equal to the second threshold value. The first perfusion index information may be identified based on an amplitude of the blood flow rate information, vibrating according to a pulse wave, and a magnitude of the blood flow rate information. The second perfusion index information may be identified based on the amplitude of the blood flow rate information, vibrating according to a pulse wave, the magnitude of the blood flow rate information, and compression interval information.

According to an example embodiment, in order to identify the blood flow rate information, the at least one processor 120 or 420 may emit light to an object via the PPG sensor 440. In order to identify the blood flow rate information, the at least one processor 120 or 420 may identify light reflected from the object. In order to identify the blood flow rate information, the at least one processor 120 or 420 may identify the blood flow rate information based on a ratio of an intensity of the emitted light to an intensity of the reflected light.

According to an example embodiment, the at least one processor 120 or 420 may further display a notification to induce an operation of the cuff of the external electronic device 505.

According to an example embodiment, the at least one processor 120 or 420 may further receive information of the external electronic device 505 for pairing, based on detecting the external electronic device 505 including the cuff. The at least one processor 120 or 420 may further transmit information of the electronic device 101, 200, or 503 to the external electronic device 505 for the pairing.

According to an example embodiment, in order to identify the compression timing information and the relaxation timing information, the at least one processor 120 or 420 may receive a signal including the compression timing information and the relaxation timing information from the external electronic device 505. The relaxation timing of the cuff may correspond to a timing at which a value corresponding to a maximum value of blood pressure measured by the external electronic device 505 becomes equal to pressure applied by the cuff.

According to an example embodiment, in order to identify the compression timing information and the relaxation timing information, the at least one processor 120 or 420 may identify timing information of an interval at which a value of the blood flow rate information does not vibrate and increases 617. In order to identify the compression timing information and the relaxation timing information, the at least one processor 120 or 420 may identify start timing information of the interval 617 as the compression timing information. In order to identify the compression timing information and the relaxation timing information, the at least one processor 120 or 420 may identify end timing information of the interval 617 as the relaxation timing information.

According to an example embodiment, the at least one processor 120 or 420 may further display a notification for guiding a user wearing the electronic device 101, 200, or 503, to wear the cuff included in the external electronic device 505 on an arm of the user, based on detecting the external electronic device 505 including the cuff.

According to an example embodiment, the first perfusion index information may be identified based on a value obtained by dividing an amplitude value of the blood flow rate information vibrating according to the pulse wave by the magnitude of the blood flow rate information. The second perfusion index information may be identified based on a maximum value—over a certain time period after the relaxation timing—of a value obtained by dividing the amplitude of the blood flow rate information vibrating according to the pulse wave by the magnitude of the blood flow rate information and further dividing by the product of the compression interval information.

As described above, according to an example embodiment, a method performed by an electronic device 101, 200, or 503 may comprise detecting an external electronic device 505 including a cuff. The method may comprise identifying blood flow rate information via a PPG sensor 440. The at least one processor 120 or 420 may comprise obtaining compression timing information of the cuff and relaxation timing information of the cuff. The method may comprise obtaining first perfusion index information, based on the blood flow rate information and the compression timing information. The method may comprise obtaining second perfusion index information, based on the blood flow rate information, the compression timing information, and the relaxation timing information. The method may comprise identifying vascular health information based on a ratio of the second perfusion index information to the first perfusion index information. The method may comprise displaying a notification indicating a vascular health state, based on the vascular health information.

According to an example embodiment, the identifying the vascular health information may comprise identifying vascular health information corresponding to a low degree of vascular health, in a case that the ratio of the second perfusion index information to the first perfusion index information is less than a first threshold value. The identifying the vascular health information may comprise identifying vascular health information corresponding to a medium degree of vascular health, in a case that the ratio of the second perfusion index information to the first perfusion index information is greater than or equal to the first threshold value and less than a second threshold value. The identifying the vascular health information may comprise identifying vascular health information corresponding to a high degree of vascular health, in a case that the ratio of the second perfusion index information to the first perfusion index information is greater than or equal to the second threshold value.

According to an example embodiment, the identifying the blood flow rate information may comprise emitting light to an object via the PPG sensor 440. The identifying the blood flow rate information may comprise identifying light reflected from the object. The identifying the blood flow rate information may comprise identifying the blood flow rate information based on a ratio of an intensity of the emitted light to an intensity of the reflected light.

According to an example embodiment, displaying a notification to induce an operation of the cuff of the external electronic device 505 may be further comprised.

According to an example embodiment, the method may further comprise receiving information of the external electronic device 505 for pairing, based on detecting the external electronic device 505 including the cuff. The method may further comprise transmitting information of the electronic device 101, 200, or 503 to the external electronic device 505 for the pairing.

According to an example embodiment, identifying the compression timing information and the relaxation timing information may comprise receiving a signal including the compression timing information and the relaxation timing information from the external electronic device 505. The relaxation timing of the cuff may correspond to a timing at which a value corresponding to a maximum value of blood pressure measured by the external electronic device 505 becomes equal to pressure applied by the cuff.

According to an example embodiment, the identifying the compression timing information and the relaxation timing information may include identifying timing information of an interval 617 at which a value of the blood flow rate information does not vibrate and increases. The identifying the compression timing information and the relaxation timing information may include identifying start timing information of the interval 617 as the compression timing information. The identifying the compression timing information and the relaxation timing information may include identifying end timing information of the interval 617 as the relaxation timing information.

As described above, according to an example embodiment, an electronic device 101, 200, or 513 may comprise at least one processor 120 or 420, a photoplethysmography (PPG) sensor 440, and a cuff. The cuff may be configured to inflate by introduction of fluid. The at least one processor 120 or 420 may identify blood flow rate information via the photoplethysmography (PPG) sensor 440. The at least one processor 120 or 420 may obtain compression timing information of the cuff and relaxation timing information of the cuff. The at least one processor 120 or 420 may obtain first perfusion index information, based on the blood flow rate information and the compression timing information. The at least one processor 120 or 420 may obtain second perfusion index information, based on the blood flow rate information, the compression timing information, and the relaxation timing information. The at least one processor 120 or 420 may identify vascular health information based on a ratio of the second perfusion index information to the first perfusion index information. The at least one processor 120 or 420 may display a notification indicating a vascular health state, based on the vascular health information.

According to an example embodiment, in order to identify the vascular health information, the at least one processor 120 or 420 may identify vascular health information corresponding to a low degree of vascular health, in a case that the ratio of the second perfusion index information to the first perfusion index information is less than a first threshold value. In order to identify the vascular health information, the at least one processor 120 or 420 may identify vascular health information corresponding to a medium degree of vascular health, in a case that the ratio of the second perfusion index information to the first perfusion index information is greater than or equal to the first threshold value and less than a second threshold value. In order to identify the vascular health information, the at least one processor 120 or 420 may identify vascular health information corresponding to a high degree of vascular health, in a case that the ratio of the second perfusion index information to the first perfusion index information is greater than or equal to the second threshold value.

According to an example embodiment, in order to identify the blood flow rate information, the at least one processor 120 or 420 may emit light to an object via the PPG sensor 440. In order to identify the blood flow rate information, the at least one processor 120 or 420 may identify light reflected from the object. In order to identify the blood flow rate information, the at least one processor 120 or 420 may identify the blood flow rate information based on a ratio of an intensity of the emitted light to an intensity of the reflected light.

According to an example embodiment, in order to identify the compression timing information and the relaxation timing information, the at least one processor 120 or 420 may identify timing information of an interval 617 at which a value of the blood flow rate information does not vibrate and increases. In order to identify the compression timing information and the relaxation timing information, the at least one processor 120 or 420 may identify start timing information of the interval 617 as the compression timing information. In order to identify the compression timing information and the relaxation timing information, the at least one processor 120 or 420 may identify end timing information of the interval 617 as the relaxation timing information.

The electronic device according to various embodiments may be one of various types of electronic devices. The electronic devices may include, for example, a portable communication device (e.g., a smartphone), a computer device, a portable multimedia device, a portable medical device, a camera, a wearable device, a home appliance, or the like. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of or all possible combinations of the items enumerated together in a corresponding one of the phrases. As used herein, such terms as “1st” and “2nd,” or “first” and “second” may be used to simply distinguish a corresponding component from another, and docs not limit the components in other aspect (e.g., importance or order). It is to be understood that if an element (e.g., a first element) is referred to, with or without the term “operatively” or “communicatively”, as “coupled with,” or “connected with” another clement (e.g., a second clement), the element may be coupled with the other element directly (e.g., wiredly), wirelessly, or via a third element.

As used in connection with various embodiments of the disclosure, the term “module” may include a unit implemented in hardware, software, or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a compiler or a code executable by an interpreter. The machine-readable storage medium may be provided in the form of a non-transitory storage medium. Wherein, the “non-transitory” storage medium is a tangible device, and may not include a signal (e.g., an electromagnetic wave), but this term does not differentiate between a case in which data is semi-permanently stored in the storage medium and a case in which the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been illustrated and described with reference to various example embodiments, it will be understood that the various example embodiments are intended to be illustrative, not limiting. It will be further understood by those skilled in the art that various modifications, alternatives and/or variations of the various example embodiments may be made without departing from the true technical spirit and full technical scope of the disclosure, including the appended claims and their equivalents. It will also be understood that any of the embodiment(s) described herein may be used in conjunction with any other embodiment(s) described herein.

What is claimed is:

1. An electronic device comprising:
memory storing instructions;
at least one processor, comprising processing circuitry; and
a photoplethysmography (PPG) sensor,
wherein the instructions, when executed by the at least one processor, individually and/or collectively, cause the electronic device to:
detect an external electronic device comprising a cuff,
identify blood flow rate information via the PPG sensor,
obtain compression timing information of the cuff and relaxation timing information of the cuff,
obtain first perfusion index information, based on the blood flow rate information and the compression timing information,
obtain second perfusion index information, based on the blood flow rate information, the compression timing information, and the relaxation timing information,
identify vascular health information based on a ratio of the second perfusion index information to the first perfusion index information, and
display a notification indicating a vascular health state, based on the vascular health information.

2. The electronic device of claim 1,
wherein, to identify the vascular health information, the instructions, when executed by the at least one processor individually and/or collectively, is configured to cause the electronic device to:
based on the ratio of the second perfusion index information to the first perfusion index information being less than a first threshold value, identify vascular health information corresponding to a low degree of vascular health,
based on the ratio of the second perfusion index information to the first perfusion index information being greater than or equal to the first threshold value and less than a second threshold value, identify vascular health information corresponding to a medium degree of vascular health, and
based on the ratio of the second perfusion index information to the first perfusion index information being greater than or equal to the second threshold value, identify vascular health information corresponding to a high degree of vascular health,
wherein the first perfusion index information is identified based on an amplitude of the blood flow rate information, vibrating according to a pulse wave, and/or a magnitude of the blood flow rate information, and
wherein the second perfusion index information is identified based on the amplitude of the blood flow rate information, vibrating according to a pulse wave, the magnitude of the blood flow rate information, and/or compression interval information.

3. The electronic device of claim 1,
wherein, to identify the blood flow rate information, the instructions, when executed by the at least one processor individually and/or collectively, is configured to cause the electronic device to:
emit light to an object via the PPG sensor,
identify light reflected from the object, and
identify the blood flow rate information based on a ratio of an intensity of the emitted light to an intensity of the reflected light.

4. The electronic device of claim 1,
wherein the instructions, when executed by the at least one processor individually and/or collectively, is configured to cause the electronic device to display a notification to induce an operation of the cuff of the external electronic device.

5. The electronic device of claim 1,
wherein the instructions, when executed by the at least one processor individually and/or collectively, is configured to cause the electronic device to:
receive information of the external electronic device for pairing, based on detecting the external electronic device including the cuff, and
transmit information of the electronic device to the external electronic device for the pairing.

6. The electronic device of claim 1,
wherein, to identify the compression timing information and the relaxation timing information, the instructions, when executed by the at least one processor individually and/or collectively, is configured to cause the electronic device to receive, from the external electronic device, a signal including the compression timing information and the relaxation timing information, and
wherein the relaxation timing of the cuff corresponds to a timing at which a value corresponding to a maximum value of blood pressure measured by the external electronic device becomes equal to pressure applied by the cuff.

7. The electronic device of claim 1,
wherein, to identify the compression timing information and the relaxation timing information, the instructions, when executed by the at least one processor individually and/or collectively, is configured to cause the electronic device to:
identify timing information of an interval at which a value of the blood flow rate information does not vibrate and increases,
identify start timing information of the interval as the compression timing information, and
identify end timing information of the interval as the relaxation timing information.

8. The electronic device of claim 1,
wherein the instructions, when executed by the at least one processor individually and/or collectively, is configured to cause the electronic device to display a notification for guiding a user wearing the electronic device, to wear the cuff included in the external electronic device on an arm of the user, based on detecting the external electronic device including the cuff.

9. The electronic device of claim 2,
wherein the first perfusion index information is configured to be identified based on a value obtained by dividing an amplitude value of the blood flow rate information vibrating according to the pulse wave by the magnitude of the blood flow rate information, and
wherein the second perfusion index information is configured to be identified based on a maximum value over a certain time period after the relaxation timing of a value obtained by dividing the amplitude of the blood flow rate information vibrating according to the pulse wave by the magnitude of the blood flow rate information and further dividing by the product of the compression interval information.

10. A method performed by an electronic device, the method comprising:
detecting an external electronic device including a cuff,
identifying blood flow rate information via a PPG sensor,
obtaining compression timing information of the cuff and relaxation timing information of the cuff,
obtaining first perfusion index information, based on the blood flow rate information and the compression timing information,
obtaining second perfusion index information, based on the blood flow rate information, the compression timing information, and the relaxation timing information,
identifying vascular health information based on a ratio of the second perfusion index information to the first perfusion index information, and
displaying a notification indicating a vascular health state, based on the vascular health information.

11. The method of claim 10,
wherein the identifying the vascular health information comprises:
based on the ratio of the second perfusion index information to the first perfusion index information being less than a first threshold value, identifying vascular health information corresponding to a low degree of vascular health;
based on the ratio of the second perfusion index information to the first perfusion index information being greater than or equal to the first threshold value and less than a second threshold value, identifying vascular health information corresponding to a medium degree of vascular health, and
based on the ratio of the second perfusion index information to the first perfusion index information being greater than or equal to the second threshold value, identifying vascular health information corresponding to a high degree of vascular health,

12. The method of claim 10,
wherein the identifying the blood flow rate information comprises:
emitting light to an object via the PPG sensor,
identifying light reflected from the object, and
identifying the blood flow rate information based on a ratio of an intensity of the emitted light to an intensity of the reflected light.

13. The method of claim 10, further comprising:
displaying a notification to induce an operation of the cuff of the external electronic device.

14. The method of claim 10, further comprising:
based on detecting the external electronic device including the cuff, receiving information of the external electronic device for pairing, and
transmitting information of the electronic device to the external electronic device for the pairing.

15. An electronic device comprising:
memory storing instructions;
at least one processor, comprising processing circuitry;
a photoplethysmography (PPG) sensor, and
a cuff configured to inflate by introduction of fluid,
wherein the instructions, when executed by the at least one processor, individually and/or collectively, cause the electronic device to:
identify blood flow rate information via the PPG sensor,
obtain compression timing information of the cuff and relaxation timing information of the cuff,
obtain first perfusion index information, based on the blood flow rate information and the compression timing information,
obtain second perfusion index information, based on the blood flow rate information, the compression timing information, and the relaxation timing information,
identify vascular health information based on a ratio of the second perfusion index information to the first perfusion index information, and
display a notification indicating a vascular health state, based on the vascular health information.
